(19) United States
(12) Patent Application Publication
(10) Pub. No.: US 2004/0016009 A1
(43) Pub. Date: Jan. 22, 2004

(54) Method of obtaining a non-human mammal susceptible to adenovirus-mediated gene delivery, a method for such delivery, and a non-human mammal susceptible to such delivery

(76) Inventors: Tiziano Tallone, Fribourg (CH); Lennart Philipson, Stockholm (SE); Sven Pettersson, Tullinge (SE)

Correspondence Address: Michael R Ward, Morrison & Foerster, 425 Market Street, San Francisco, CA, 94105-2482, US

(21) Appl. No.: 10/363,973

(22) PCT Filed: Sep. 6, 2001
(86) PCT No.: PCT/SE01/01896

(51) Int. Cl. A01K 67/027; A61K 48/00; C12N 15/861

(52) U.S. Cl. 800/18; 424/93.21; 800/21; 435/456

(57) ABSTRACT

A method of obtaining a non-human mammal susceptible to adenovirus-mediated gene delivery, a method for such delivery, and a transgenic non-human mammal susceptible to adenovirus-mediated gene delivery, and more specifically a trans-genic mouse that expresses a cytoplasmically truncated human Coxsackievirus and Adenovirus Receptor (hCAR) in essentially all tissues thereof. The mammal allows for efficient infections at low multiplicity of infection (MOI) into cells that are normally resistant or not very susceptible to adenovirus-mediated gene delivery, such as spleenocytes and dendritic cells (DC). The hCAR transgenic mammal is highly susceptible to adenovirus-mediated gene transfer and will be a useful tool to probe gene function in development and to elucidate molecular pathways, dynamic properties and differentiation mechanisms in non-transformed cells.

### TECHNICAL FIELD OF THE INVENTION

The present invention relates to a method of obtaining a non-human mammal susceptible to adenovirus-mediated gene delivery, a method for such delivery, and a transgenic non-human mammal susceptible to adenovirus-mediated gene delivery, and more specifically a transgenic mouse that expresses a cytoplasmically truncated human Coxsackievirus and Adenovirus Receptor (hCAR) protein in essentially all tissues thereof. The mouse allows for efficient infections at low multiplicity of infection (MOI) into cells that are normally resistant or not very susceptible to adenovirus-mediated gene delivery, such as, for example, spleenocytes and dendritic cells PC). The hCAR transgenic mice of the present invention are therefore highly susceptible to adenovirus-mediated gene transfer and will be a useful tool to probe gene function in development and to elucidate molecular pathways, dynamic properties and differentiation mechanisms in non-transformed cells.

### BACKGROUND ART

The elucidation of biological pathways is often derived from the experimental observations following introduction or deletion of nucleic acids. Such experiments with mammalian organisms or cells can be complex and time-consuming. For example, the engineering of in vivo chromosomal alterations usually takes about two years before phenotypes can be assessed, whilst at the same time many primary eukaryotic cells remain recalcitrant to transfection. The use of recombinant viruses is currently one of the most powerful ways to introduce foreign genes into mammalian cells in vivo and in vitro. Adenoviruses (Ads) have received considerable attention as gene delivery vectors because of i) their relatively large cloning capacity, ii) ease of genetic manipulation and growth to high virus titers, iii) the structural stability of the virus particle and iv) their ability to infect proliferating and quiescent cells. Despite the fact that Ad can infect a wide range of cell types, some tissues and cells, such as lymphocytes, are refractory to adenovirus infection.

The CAR is a 46 kDa transmembrane protein that belongs to the immunglobulin surperfamily and possesses the highest structural similarity to CTX and human A33-antigen. Its cellular function has not yet been completely elucidated, but recent data suggest that CAR may function as an adhesion molecule. The expression pattern of CAR varies, not only between different developmental stages and tissues but also between species. While CAR is abundantly expressed in the majority of the mouse epithelial cells during embryogenesis, its expression in adult mouse is restricted to a few epithelial cells (Tomko, R. P. et al., Exp. Cell Res. 255, 47-55 (2000) and Fechner, H. et al., Gene Ther. 6, 1520-1535 (1999)).

Previous work by Leon, R. P. et al., Proc. Natl. Acad. Sci. USA 95, pp. 13159-13164 (1998) and Wang, X. & Bergelson, J. M., J. Virol. 73, 2559-2562 (1999), has indicated that the cytoplasmic portion of CAR is not required for virus infection.

WO9833819 generally relates to human CAR and murine CAR protein molecules, and peptide fragments of these proteins, corresponding, for example, to the extracellular domains and other functional derivatives. The isolated proteins and fragments are used to prevent or treat virus infections. The expression of the DNA encoding these virus receptors in cells lacking said receptors, is said to render the cells susceptible to transformation by adenoviral vectors carrying genes for gene therapy. The hCAR and mCAR has also been shown to be expressed to various extents in a number of cell types. A transgenic non-human eukaryotic animal (preferably a rodent, such as a mouse), essentially all of whose germ cells and somatic cells contain genomic DNA encoding the hCAR or mCAR protein or a functional derivative thereof, capable of serving as a human Ad2, Ad5 or CVB virus receptor, is also envisaged.

Accordingly, it is an object of the present invention to provide method for obtaining a non-human mammal, substantially all of whose tissues are susceptible to adenovirus-mediated gene transfer, suitable for use as an animal model and for validating the functions of human genes. Different cell types and tissues of said mammal should preferably also exhibit comparable capabilities of such gene transfer.

For the purpose of the present invention, a suitable animal model, should be susceptible to adenovirus-mediated gene transfer, with a minimum of, and preferably without any, interfering biological responses mediated by the receptor itself. Accordingly, the receptor should ideally be essentially biologically inactive, or “dead”, i.e. only mediating the internalisation of the adenovirus, carrying the gene being transferred, without initiating any other biological signals.

The above object has been achieved by means of the method of claim 1, involving the steps of (a) providing an expression vector containing the human ubiquitin C promoter linked to the gene encoding the hCAR receptor lacking its cytoplasmic tail, (b) introducing the vector into a fertilised oocyte or an embryonic stem cell of the mammal,

According to another aspect of the present invention, a method of adenovirus-mediated gene delivery to a non-human mammal is provided, wherein a gene contained in an adenovirus vector is delivered to a mammal expressing the hCAR protein lacking its cytoplasmic tail.

According to a further aspect a non-human mammal expressing hCAR lacking its cytoplasmic tail is also provided, said mammal being obtainable by means of the method of claim 1.

According to yet a further aspect of the present invention, an expression vector for use in the method of claim 1 is also provided, containing the human ubiquitin C promoter linked to the gene encoding the hCAR protein lacking its cytoplasmic tail.

Further embodiments and advantages of the present invention will be evident from the following description and appended claims.

### SUMMARY OF INVENTION

According to the method of the present invention, a mammal model is obtained expressing comparable levels of truncated hCAR in substantially all tissues thereof, and also at comparable levels among the different cell types. Such mammal will for example provide an exemplary animal model and a valuable tool in tests and studies involving validation of the function of a transferred gene. The value of the mammal is further enhanced by the relatively stable expression of hCAR obtainable according to the present invention.

According to one embodiment of the present invention a transgenic mouse strain is provided, which strain contains human CAR (hCAR) lacking its cytoplasmic tail. The mice are highly susceptible to Ad-mediated gene transfer. Importantly, spleenocytes and dendritic cells (DC) that are normally resistant or not very susceptible to Ad-mediated gene delivery were readily infected at low MOI.

These mice allow for efficient infections at low MOI (multiplicity of infection) into cells that are normally resistant or not very susceptible to adenovirus-mediated gene delivery, such as spleenocytes and dendritic cells (DC). CAR transgenic mice are therefore highly susceptible to adenovirus-mediated gene transfer and will be a useful tool to probe gene function in development and to elucidate molecular pathways, dynamic properties and differentiation mechanisms in non-transformed cells.

According to the present invention, it has surprisingly been found that by using the gene encoding truncated hCAR, i.e. the hCAR protein lacking its cytoplasmically portion, driven by the recently discovered ubiquitin C promotor, expression of an essentially biologically inactive form of the receptor, i.e., except for the ability of uptake of adenovirus, can be obtained in an animal model transfected with a genetic construct comprising said gene and promotor. Such animal was surprisingly found to express the truncated receptor in all tissues examined. This is of great importance for the use of such an animal as an animal model in, for example, the validation of the function of a human gene. The levels of expression among different types of tissues have also been found to be comparable, or reasonably uniform, such as seemingly equal, which is another valuable characteristic for the purpose of the present invention. Moreover, as opposed to the expression of the natural full-length endogenous receptor, the expression of the truncated receptor of the invention has not been found to be temporally regulated.

Furthermore, due to the biological inactivity of the receptor, the animal can also be made to express a gene which otherwise, for example, could trigger apoptosis or would be lethal to the animal or any cell types thereof.

The stability of expression of the biologically inactive form of the hCAR protein in the mammal can be further enhanced by means of the inclusion of an intron sequence from β-globin downstream of the gene encoding the hCAR protein lacking its cytoplasmically tail in the expression vector used for obtaining the animal.

### BRIEF DESCRIPTION OF THE DRAWINGS

FIG. 1 depicts the structure of the transgene construct of the present invention.

FIG. 2a and b show hCAR expression patterns in different organs from a transgenic mouse of the invention.

FIG. 3 illustrates the effect of injection of recombinant adenovirus expressing a green flourescent protein into a transgenic mouse of the invention (3a, b, c and d) as compared to a control animal (3e, f, g and h).

FIG. 4 shows expression of transgenic hCAR in spleenocytes and dendritic cells.

FIG. 5a shows infection of spleenocytes and 5b shows infection of dendritic cells.

### DETAILED DESCRIPTION OF THE PRESENT INVENTION

With reference to FIG. 1, the transgene construct for use in the method of the present invention is shown. Outlined above is the truncated human CAR, in which SP is a signal peptide; IG1 and IG2, immunoglobulin-like domain 1 and 2, respectively; TM, the transmembrane-spanning region. The truncated hCAR contains only four amino acids (CRKK) C terminal to the trans-membrane domain. Below in FIG. 1 is a schematic map of the pβUbiC-hCAR(1-262) plasmid used in the method of the present invention. The human ubiquitin C promoter and the truncated human CAR are shown as open boxes. The black box and the thick line denote the rabbit β-globin sequences, as indicated. The poly-adenylation signal is indicated as a black dot.

The present invention will now be described in further detail in the following examples.

### EXAMPLES

Procedures and Materials Used

Cell Cultures

Cos and HER 911 cells were cultured in DMEM supplemented with 10% FCS (Gibco BRL), 2 mM glutamine (Gibco BRL) and 100 U/ml penicillin/streptomycin (Gibco BRL). Cos cells were transfected with the polyethylenimine reagent (PEI, 25 kDa, Merk) as described by Boussif, O. et al. in Proc. Natl. Acad. Sci. USA 92, 7297-7301 (1995).

The mouse thymoma cell line EL-4 was cultured in RPMI supplemented as described for the Cos and HER 911 cells. Primary lymphocytes were grown in RPMI supplemented with 15% FCS (Gibco BRL), 2 mM glutamine (Gibco BRL) and 100 U/ml penicillin/streptomycin (Gibco BRL) and 50 μM 2-mercaptoethanol (Sigma). Dendritic cells (DC) were generated by culture of bone marrow cells in presence of GM-CSF and IL-4 as described by Inaba, K. et al., in J. Ex. Med. 176, 1693-1702 (1992). Briefly, bone marrow cells were collected, washed with PBS and placed in 12 well plates (3.0×10⁶ cells/3 ml/well) in DMEM medium supplemented with 15% FCS (Integro b.v., USA), 2 mM glutamine (Gibco BRL), 100 U/ml penicillin/streptomycin (Gibco BRL) and 10 ng/ml each of mouse GM-CSF and mouse IL-4 (both from PreproTech EC, London, GB). Two-thirds of the medium were replaced on day 2, 4 and 6. On day 7, adherent DCs were harvested and 3.0×10⁶ cells were replated in 6 cm petri dishes with supplemented DMEM containing GM-CSF and IL-4. 36 hours later, DCs were infected with AdGFP, as win be further explained below in Example 2.

Western Blot

Tissues were homogenised in 1% deoxycholate, 1% triton x-100 and protease inhibitors (complete™, Boehringer Mannheim) for 1 hour at 4° C. and centrifuged at 20,000 g for 15 minutes. The supernatant was analysed by SDS/10% PAGE without heating or the addition of reducing agents using the discontinuous buffer system. After transfer to poly(vinylidene diflouride) membranes the blots were probed with the anti-IG1 (Tomko, R. P. et al.; Exp. Cell Res. 255, 47-55 (2000)) or the RmcB anti-bodies for 1 h and visualised by addition of horseradish peroxidase secondary anti-bodies and the ECL detection system (Amersham). The rabbit anti-IG1 antibody was raised against a GST fusion protein containing the -immunoglobulin-like domain 1 (IG1) of the CAR.

Work with the recombinant adenovirus expressing the green fluorescence protein (AdGFP)

Adenovirus expressing the GFP gene and potentiated by the CMV promoter was amplified in HER 911 cells and purified by CsCl centrifugation as described by Fallaux, F. J. et al., Hum. Gene Ther. 7, 215-222 (1996) and Precious, B. and Russell, W. C., in Mahy, B. W. J. (ed.), Virology: a practical approach IRL Press, Oxford, 1985, pp. 192-205. The end-point cytopathic effect assay (Precious and Russel, cited above) was used to determine a titer of 1.4×10¹⁰ cpe units/ml. Experiments with spleenocytes or DCs were performed with heterozygous transgenic mice 8 to 10 weeks old. Spleenocytes were infected as follows: after removal of erythrocytes, the remaining single cell suspension were washed twice with PBS and cultured in RPMI medium with 30 nM PMA (Sigma, USA). After 36 hours, cells were harvested and washed once with PBS. Typically, 1.4×10⁶ cells in 200 μl RPMI were transferred to a 5 ml polystyrene round-bottom tube where the designated amount of AdGFP was added. The tube was gently agitated for 25 min. at room temperature followed by 25 more min. at 37° C. with occasional shaking. Finally, the infected cells were cultured in the supplemented RPMI for an additional 36 hours before evaluation for GFP expression by FACS analysis. In vitro maintained DCs were infected on day 8-9 as follows: the cells were washed with PBS, covered with 1 ml of DMEM and mixed with the designated amount of AdGFP. After 25 min. of gentle agitation at room temperature, and 25 min. at 37° C., the virus was removed and cells were cultured in DCs medium for an additional 36 hours before FACS analysis.

Flow Cytometry Analyses

The following antibodies were used: PE-anti-mouse-CD45/B220 and PE-anti-mouse-CD11c (both from Pharmingen, USA). The cells were analysed using a FAC-Scahibur device and Cell Quest software version 3.1 f (both from Becton Dickinson, USA).

### Example 1

PCR and Southern blot analyses were made according to standard procedures. The transgene gene construct pβUbiC-hCAR(1-262) containing the human Ubiquitin C promoter (position-1225 to -6, (Schorpp, M. et al., Nucleic Acids Res. 24, 1787-1178 (1996))) was linked to the hCAR receptor (amino acid 1 to 262) gene that lacks its cytoplasmic tail. As previously mentioned, the latter is not required for efficient Ad infections. pβUbiC-hCAR(1-262) contains a rabbit β-globin splice/polyadenylation signal from the pSCT expression vector. The transgene was cut with Xho I and Sph I, purified and injected into fertilised oocytes as described previously by Arulampalam, V., Grant, P. A., Samuelsson, A., Lendahl, U. and Pettersson, in S., Eur. J. Immunol. 24, 1671-1677 (1994).

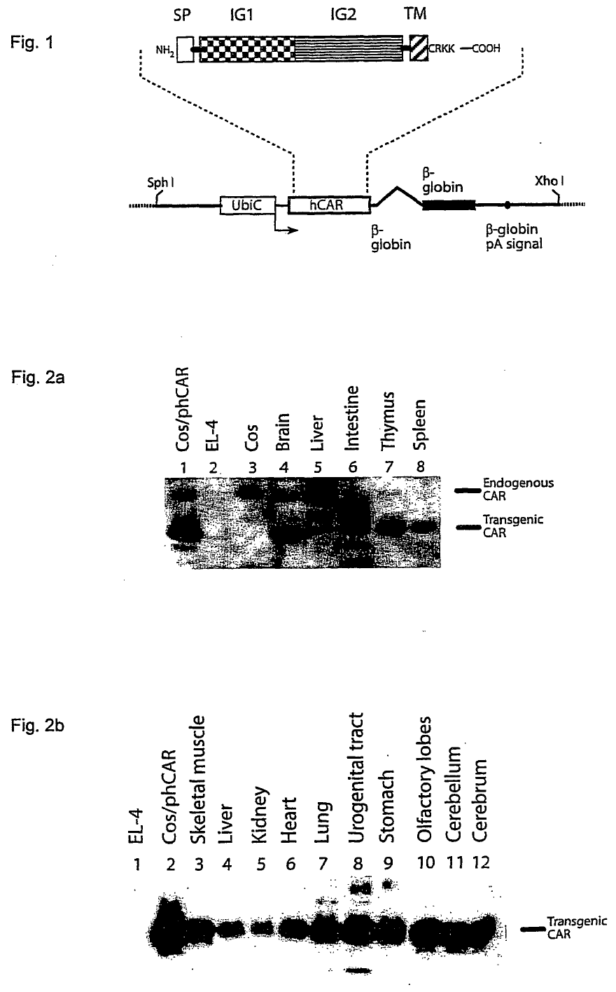

Mice carrying the truncated hCAR were initially screened by PCR and several independent transgenic founders were identified. Male founders were used to derive seven independent transgenic lines. The progeny generated by these mice was analysed by FACS analysis and Western blots for hCAR expression. A line expressing high levels of transgenic hCAR in all tissues examined was selected for further experimentation. In FIG. 2a and b, the expression of hCAR in different tissues of this line is shown. This line was used in all further experiments. The high levels of expression was also confirmed by Southern blot (not shown). These animals appear healthy and have no obvious defects.

In the Western blot of hCAR, shown in FIG. 2a, rabbit antibodies raised against a GST fusion protein containing the immunoglobulin-like domain 1 (IG1) of the CAR were used. They recognise both endogenous and human transgenic CAR. Arrows indicate the signals corresponding to the endogenous or transgenic hCAR. Lane 1 is a positive control of Cos cells transfected with the hCAR expression plasmid pβUbiC-hCAR(1-262). Lane 2 is a negative control of the CAR deficient EL4 mouse thymoma cell line. Lane 3 are Cos cells transfected with empty expression vector.

The expression patterns of the hCAR transgene in different tissues were also analysed by Western blot using a monoclonal antibody (RmcB) that is specific for the transgenic hCAR. The arrow indicates the signal corresponding to the transgenic hCAR. Lane 1 and 2 are as described in FIG. 2a.

### Example 2

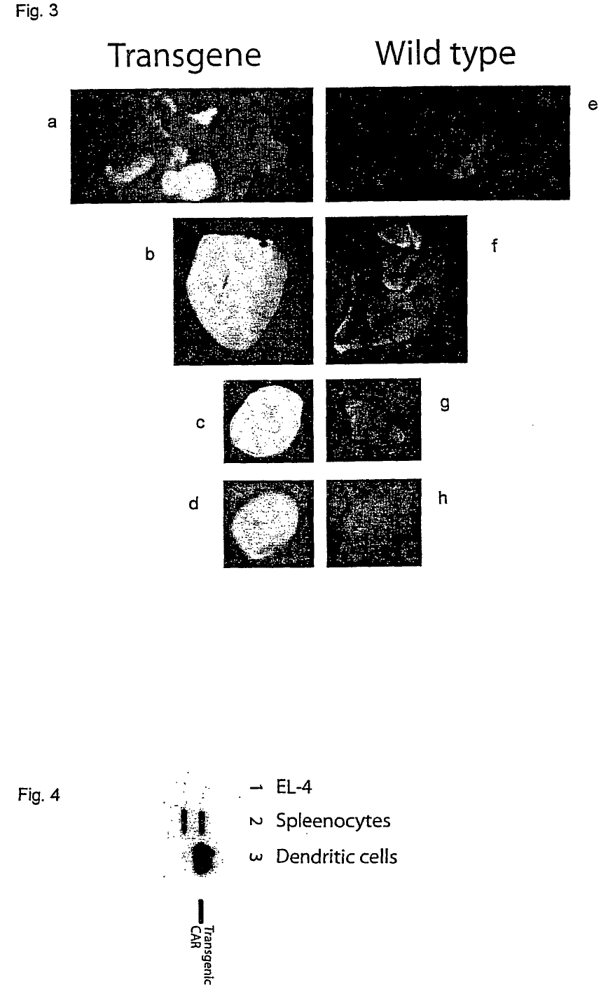

To assess whether the transgenic hCAR mice displayed increased susceptibility to Ad-mediated gene delivery, 10⁸ cpe units of a recombinant Ad expressing the green fluorescent protein AdGFP was injected in the peritoneal cavity of a transgenic mouse (a, b, c and d) and a negative littermate (e, f, g and h). After 24 hours, the GFP expression levels in the peritoneal cavities were monitored with a fluorescence image analyser system as depicted in FIG. 3a and 3e. A considerably enhanced susceptibility towards Ad-mediated gene delivery of the transgenic mouse was observed as compared to the negative littermate. Furthermore, whole organs were assessed for GFP expression; the transgenic liver shown in FIG. 3b, the transgenic omentum containing fat tissue shown in FIG. 2c, and the transgenic urinary bladder shown in FIG. 2d, were much more fluorescent than corresponding organs of the control mouse, shown in FIG. 3f, g and h, respectively. Thus, the broad expression of the transgenic hCAR strongly improves the in vivo efficiency of Ad-mediated gene delivery in many different tissues.

### Example 3

Primary B lymphocytes are generally known to be very resistant to most currently available gene transfer techniques, including adenoviral vectors. These primary cells therefore provide an instructive medium in which to assess the extent that the hCAR transgene increases Ad virus gene delivery.

The presence of the hCAR protein in the spleenocytes of transgenic animals was examined by Western blot, shown in FIG. 4. Expression levels of the hCAR transgene in spleenocytes and DCs was confirmed by Western blot with the RmcB antibody. In FIG. 4, lane 1 is negative control: the CAR deficient EL-4 mouse thymoma cell line, lane 2 is spleenocytes and Lane 3 is dendritic cells.

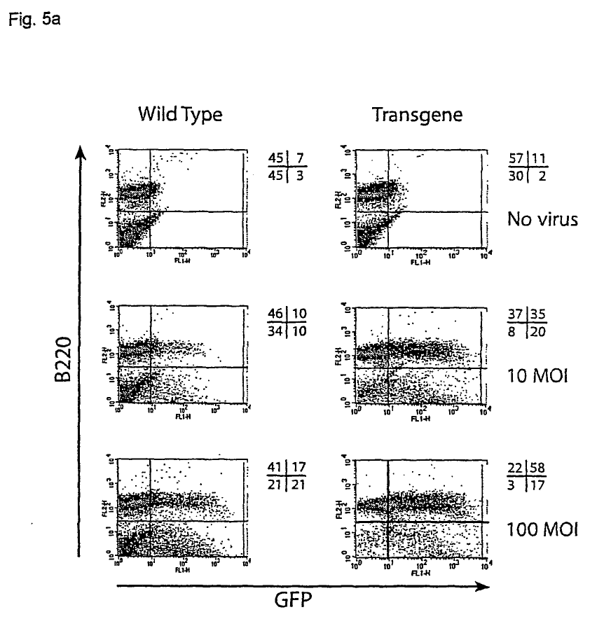

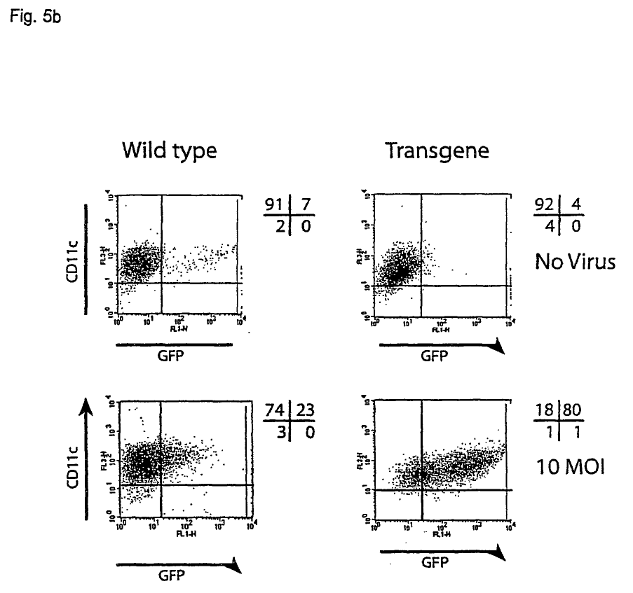

Single cell suspensions of spleenocytes obtained from a trangenic mouse and a negative littermate were stimulated with PMA and infected with different quantities of AdGFP (MOI 0, 10 or 100). After 36 hours, the live cells were analysed for the expression of GFP and for the presence the lymphocyte marker B220 by flow cytometry. Plots on the left-hand side refer to the negative littermate, plots on the right-hand side refer to the hCAR transgenic mouse. The percentage of cells present in each quadrant is indicated. As seen in FIG. 5a, transduction of the transgenic cells was much more efficient over the two MOIs tested, allowing a significant population of cells to express high levels of GFP. In particular, at a MOI of 100, the density plot FACS analysis suggested that the entire population of transgenic cells appears to have shifted to the right implying that nearly all cells were transduced. Accordingly, the expression of the transgenic hCAR in spleenocytes and dendritic cells confers enhanced susceptibility to Ad transduction.

Dendritic cells have recently been considered as a candidate cell type to use for immunisation protocols such as vaccination, tolerance and anti-tumour immunity Kirk, C. J. & Mulé, J. J., Hum. Gene Ther. 11, 797-806 (2000). Given the relatively unstable phenotype of DCs, however, non-perturbing methods of gene delivery have to be employed to maintain viability and immuno-stimulatory capacity. Moreover, mature DCs are relatively resistant to Ad mediated gene delivery and high viral titers (MOI>100) are required to achieve efficient gene transfer. This is explained by the fact that mature DCs do not express CAR and express very low levels of αᵥ-integrins.

The detection of the transgenic hCAR on in vitro cultured mature DCs (FIG. 4, lane 3) prompted the inventors to assess whether transgene receptor expression would provide improved and more efficient Ad-mediated gene delivery.

Mature in vitro generated transgenic and control DCs obtained from bone marrow cultures (see General procedures) were infected with AdGFP at approximately 10 MOI. After 36 hours, live cells were analysed by FACS for the expression of GFP and for the presence of the mature DC marker CD11c. In FIG. 5, on the left-hand side the negative littermate is shown, and on the right-hand side, the hCAR transgenic mouse. The quadrant statistics are indicated. As can be seen from FIG. 5b, approximately 80% of the transgenic DC were highly double positive, compared to 23% of the control cells. Furthermore, the entire population of transgenic DCs appeared to have shifted to the right, suggesting that all cells may have been transduced. According to Kirk, C. J. & Mulé, J. J., cited above, approximately 80% of normal mature DCs can be infected but with MOIs of 100 or higher. Thus, the experiments with DCs of the present invention that express hCAR demonstrate that similar results can be obtained with at least 10-fold less virus particles. This may be an important consideration when performing reconstitution experiments with DCs in vivo. Furthermore, the previously reported host inflammatory responses that are directed against recombinant Ad-vectors may be reduced by the use of lowered virus titers, enabled by means of the present invention.

Results and Discussion

The problems associated with efficient introduction of genes into primary cells limits the characterisation of cellular pathways that control cell growth, differentiation, and death. As a new approach to gene transfer, a mouse model has been developed that allows efficient delivery of genes with Ad vectors. These mice are believed to be a valuable tool for a variety of applications that concern the functional analysis of genes in vitro and in vivo under normal and pathological conditions and especially to test the efficacy of proposed gene therapy procedures.

One major goal in the field of gene therapy is the development of Ad vectors that target a gene to a specific cell type or organ. This requires both the introduction of tissue specific ligands and the abrogation of Ad to its cognate receptor. Ideally, such tissue specific Ad would allow the systemic administration of Ad vectors in a restricted fashion in vivo (Wickham, T. J., Gene Ther. 7, 110-114 (2000)). The hCAR transgenic mice may provide an ideal test system in which to assess whether the retargeted Ad vector avoids interaction with CAR and homes to its newly generated receptor, thereby selectively localising gene expression to the tissue of interest.

In some cell types CAR expression is polarised. For example, in differentiated, ciliated human airway epithelial cells, CAR was found to be confined to the basolateral membrane (Walters, R. W. et al., J. Biol. Chem. 274, 10219-10226 (1999)). It has recently been reported that both tailless and GPI-anchored CAR appear on the apical surfaces of polarised cells (Pickles, R. J., Fahrner, J. A., Petrella, J. M., Boucher, R. C. & Bergelson, J. M., J. Virol. 74, 6050-6057 (2000)), consistent with the current model which suggests that protein sorting to the basolateral membrane is determined by signals within the cytoplasmic domain (Mostov, K. E. & Cardone, M. H., BioEssays 17, 129-138 (1995)). Since our transgenic mouse express a CAR lacking the cytoplasmic domain, it is tempting to speculate that the transgenic receptor could be localised on the apical surfaces of polarised cells. This could be an additional advantage that will facilitate Ad entry into ciliated epithelial cells.

Moreover, it is clear that parameters, in addition to CAR, may contribute to an efficient Ad-gene delivery. The broad transgene expression of hCAR could open for a new set of additional experiments. Factors like the glycosylation grade of other cell surface proteins (Pickles, R. J., et al., cited above), physiological features of some organs such as the pH, the route of vector application and anatomical barriers (Kass-Eisler, A. et al., Gene Ther. 1, 395-402 (1994)) could be evaluated through the use of the transgenic hCAR mice.

The possibilities offered by these transgenic mice to deliver Ad vectors to many different primary cells and tissues at high efficiency will be useful for many applications related to the functional analysis of specific genes. For instance, the mice could be particularly attractive for gain of function experiments or for introducing Ad-mediated dominant mutations, such as the expression of ribozymes or anti-sense RNA and the expression of trans-dominant negative molecules in specific tissues and cells. These applications provide alternatives to the classical time- and resources-consuming transgenic approach.

Alternatively, by crossing the hCAR mice with mouse lines that have specific genes inactivated, it may be possible to test for rapid gain of function experiments or to evaluate the introduction of an alleviating factor and its effect on a particular phenotype. Such readouts may help to dissect, at the molecular level, the cellular pathways under investigation and could serve as a rapid screen for gene therapy candidates.

In a different but associated approach, crossing the hCAR transgenic mice with conditionally targeted animals will allow the efficient introduction of Ad vectors expressing the Cre recombinase into specific tissues, thereby achieving temporal and spatial control of gene mutations. A further advantage offered by the hCAR transgenic mice is that multiple gene products could be introduced simultaneously by co-transduction with different Ad vectors. By varying the MOI of the different vectors, the expression levels of the various genes can be controlled.

Finally, the recently developed “PEI-DNA-adenovirus” technique (Baker, A. et al., Gene Ther. 4, 773-782 (1997)) has made it possible to deliver genes into previously refractory cells. This method takes advantage of synthetic polycation polyethylenimine (PEI) to condense plasmid DNA into small PEI-DNA molecules. As a result, these positively charged complexes will bind ionically to the negative charged adenovirus capsid and will be delivered to cells through the Ad infectious route. This method avoids the problems associated with viral gene expression as the Ad is inactivated by psoralen (Cotten, M. et al., Virology 205, 254-261 (1995)). This technique, in combination with primary cells obtained from our hCAR mice, should provide the opportunity to deliver plasmids to cultures of any primary cell type and it may therefore now be possible to efficiently transfect several different types of primary cells with conventional plasmid vectors in vitro. The advantages of this method are obvious and there appears to be numerous potential applications.

1. Method of obtaining a non-human mammal exhibiting stable expression of truncated hCAR in substantially all tissues and susceptible to adenovirus mediated gene transfer, including the following steps:
(a) providing an expression vector containing the human ubiquitin C promoter linked to the gene encoding the hCAR protein lacking its cytoplasmic tail,
(b) introducing the vector into a fertilised oocyte or an embryonic stem cell of the mammal.

2. Method of claim 1, wherein the expression vector also contains an intron sequence from β-globin downstream of the gene encoding hCAR protein lacking its cytoplasmically tail.

3. Method of any of the previous claims, wherein the mammal is a mouse.

4. Method of adenovirus-mediated gene delivery to a non-human mammal, wherein a gene contained in an adenovirus vector is delivered to a mammal expressing the hCAR protein lacking its cytoplasmic tail.

5. Method of claim 4, wherein two or more different genes are delivered by means of two or more different adenovirus vectors containing said genes.

6. Method of claim 4 or 5, wherein the adenovirus vector or vectors is injected into the blood circulatory system, a desired organ, or body tissue, of the mammal.

7. Method of any of the claims 4-6, wherein the mammal is a mouse.

8. Method of any of the claims 4-7, wherein the mammal is obtained by means of the following steps:
(a) providing an expression vector containing the human ubiquitin C promoter linked to the gene encoding the hCAR protein lacking its cytoplasmic tail,
(b) introducing the vector into a fertilised oocyte or an embryonic stem cell of the mammal.

9. Non-human mammal expressing the hCAR protein lacking its cytoplasmic tail, obtainable by means of the method of claim 1.

10. Non-human mammal of claim 9, wherein the mammal is a mouse.

11. Expression vector for use in the method of claim 1, containing the human ubiquitin C promoter linked to the gene encoding the hCAR protein lacking its cytoplasmic tail.
